(19) United States
(12) Patent Application Publication
(10) Pub. No.: US 2016/0199220 A1
(43) Pub. Date: Jul. 14, 2016

(54) BODY COOLER AND BODY WARMER

(71) Applicant: DAISAKU SHOJI LTD., Tokyo (JP)

(72) Inventor: Ippei DAISAKU, Tokyo (JP)

(73) Assignee: DAISAKU SHOJI LTD., Tokyo (JP)

(21) Appl. No.: 14/912,880

(22) PCT Filed: Aug. 20, 2013
(86) PCT No.: PCT/JP2013/072152

§ 371 Date: Feb. 18, 2016

(51) Int. Cl. A61F 7/02

(52) U.S. Cl. CPC A61F 7/02; A61F 2007/0011

(57) ABSTRACT

A body cooling tool that can be used to cool any portion of a body and can be easily used at any time is provided. A body warming tool that can realize a high heat retaining property is provided.

A body cooling tool has a cooling member being a narrow long shape and having a water-absorbent material 11; and an elastic member 20 provided at an end portion of the cooling member 10. When the body cooling tool is wound around the predetermined portion of the body, the cooling member 10 is pressed against the predetermined portion of the body by elastic force of the elastic member 20, and the density of the water-absorbent material is increased so that cooling efficiency is enhanced.

### TECHNICAL FIELD

The present invention relates to a body cooling tool that is wound around a predetermined portion of a body and cools the portion, and a body warming tool that is wound around a predetermined portion of a body and warms the portion.

### BACKGROUND ART

Conventionally, body cooling tools for cooling a body are known. As an example, a head portion cooling tool is disclosed (JP 2008-104694 A), which is configured from a frontal region storage pocket provided at a position coming to a frontal region, an occipital region portion storage pocket connected with the frontal region storage pocket with a band portion and provided at a position coming to an occipital region, an upper cervical spine storage pocket coupled to the occipital region portion storage pocket with coupling means and provided at a position coming to an upper cervical spine, a cervical spine storage pocket coupled to the upper cervical spine storage pocket with coupling means and provided at a position coming to a cervical spine, and a catching band that catches the cervical spine storage pocket to a neck, where the respective storage pockets store cold insulators with insertion slots being tilted.

However, the cooling tool disclosed in JP 2008-104694 A is used to cool the head portion, and its use is limited. In addition, the cooling tool disclosed in JP 2008-104694 A stores the cold insulators and is used, and is supposed to be used after the cold insulators are cooled. Therefore, the cooling tool cannot be easily used at any time.

### SUMMARY OF INVENTION

### Problem to be Resolved by Invention

The present invention has been made in view of the foregoing, and provides a body cooling tool that can be used to cool any portion of a body and can be easily used at any time . Further, the present invention provides a body warming tool that can realize a high heat retaining property.

### Solution to Problem

The first aspect according to a body cooling tool of the present invention is the body cooling tool which is wound around a predetermined portion of a body and which cools the portion comprising:

a cooling member being a narrow long shape and having a water-absorbent material; and

an elastic member provided at an end portion of the cooling member,

wherein the water-absorbent material is expanded when moisture is contained in the water-absorbent material, and when the moisture contained in the water-absorbent material is vaporized, heat is drawn so that a cooling function is realized, and

wherein when the body cooling tool is wound around the predetermined portion of the body, the cooling member is pressed against the predetermined portion of the body by elastic force of the elastic member, and the density of the water-absorbent material is increased so that cooling efficiency is enhanced.

The first aspect according to the body cooling tool of the present invention further may comprise a bag portion including the water-absorbent material therein,

wherein the water-absorbent material may be a water-absorbent polymer.

In the first aspect according to the body cooling tool of the present invention,

the water-absorbent material may be water-absorbent cloth.

In the first aspect according to the body cooling tool of the present invention,

the elastic member may be formed into a band shape with a width of 5 mm or more.

In the first aspect according to the body cooling tool of the present invention,

the elastic member may be freely attachable/detachable to/from the cooling member.

In the first aspect according to the body cooling tool of the present invention,

when predetermined pulling force is added, the elastic member may be detached from the cooling member.

In the first aspect according to the body cooling tool of the present invention,

an attaching position of the elastic member with respect to the cooling member or an attaching position of the cooling member with respect to the elastic member may be able to be changed.

In the first aspect according to the body cooling tool of the present invention,

the length of the elastic member or the length of the cooling member may be freely adjustable.

In the first aspect according to the body cooling tool of the present invention,

a first cooling member attaching portion may be provided at one end of end portions of the cooling member, and a second cooling member attaching portion is provided at the other end of the end portions of the cooling member,

wherein a first elastic attaching portion may be provided at one end of end portions of the elastic member, and a second elastic attaching portion is provided at the other end of the end portions of the elastic member,

wherein the first cooling member attaching portion may be freely attachable/detachable to/from the first elastic attaching portion, and the second cooling member attaching portion is freely attachable/detachable to/from the second elastic attaching portion, and the first cooling member attaching portion is freely attachable/detachable to/from the second cooling member attaching portion.

The first aspect according to the body cooling tool of the present invention may further comprise an auxiliary elastic member having a different length from the elastic member.

In the first aspect according to the body cooling tool of the present invention,

a notch may be provided at a predetermined portion of the cooling member, and the bag portion may be able to be bent with the notch in a direction perpendicular to the longitudinal direction of the cooling member, and

the cooling member may cool at least the back side of the neck of the human body, and may be able to pass above ears of the human body.

In the first aspect according to the body cooling tool of the present invention,

coupling tools maybe provided at a predetermined portion of the cooling member, and the two places of the cooling member may be coupled with the coupling tools so that the bag portion may be able to be bent in the direction perpendicular to the longitudinal direction of the cooling member, and

the cooling member may cool at least the back side of the neck of the human body, and may be able to pass above ears of the human body.

The second aspect according to the body cooling tool of the present invention is the body cooling tool which is wound around a predetermined portion of a body and which cools the portion comprising:

a first cooling member being a narrow long shape and having a first water-absorbent material; and

a second cooling member being a narrow long shape and having a second water-absorbent material; and

an elastic member being freely attachable/detachable to/from both ends of the first cooling member, and being freely attachable/detachable to/from both ends of the second cooling member;

wherein each the first water-absorbent material and the second water-absorbent material is expanded when moisture is contained in the first water-absorbent material and the second water-absorbent material, and when the moisture contained in the first water-absorbent material and the second water-absorbent material is vaporized, heat is drawn so that a cooling function is realized,

wherein an end of the first cooling member is also freely attachable/detachable to/from both sides of the first cooling member,

wherein any of following types of usage can be selected: usage of attaching both ends of the elastic member to both ends of the first cooling member; usage of the attaching the both ends of the elastic member to both ends of the second cooling member; and usage of attaching the one end of the first cooling member to the other end of the second cooling member, attaching the other end of the first cooling member to the other end of the elastic member, and attaching the one end of the second cooling member to the one end of the elastic member,

wherein when the body cooling tool is wound around the predetermined portion of the body, the first cooling member, the second cooling member or the both of the first cooling member and the second cooling member is pressed against the predetermined portion of the body by elastic force of the elastic member, and the density of the water-absorbent material is increased so that cooling efficiency is enhanced.

In the second aspect according to the body cooling tool of the present invention,

the density of the first water-absorbent material of the first cooling member may be higher in the one end side of the first cooling member than the other end side,

the density of the second water-absorbent material of the second cooling member may be higher in the other end side of the second cooling member than the one end side,

in the usage of attaching the one end of the first cooling member to the other end of the second cooling member, attaching the other end of the first cooling member to the other end of the elastic member, and attaching the one end of the second cooling member to the one end of the elastic member, the density of the first water-absorbent material of the first cooling member in the end portion at the opposite side to the elastic member may be made higher than the density of the first water-absorbent material of the first cooling member in the end portion at the elastic member side, and the density of the second water-absorbent material of the second cooling member in the end portion at the opposite side to the elastic member may be made higher than the density of the second water-absorbent material of the second cooling member in the end portion at the elastic member side.

A body warming tool according to the present invention is the body warming tool which is wound around a predetermined portion of a body and which warms the portion comprising:

a warming member having a heat retaining property; and

an elastic member provided at an end portion of the warming member,

wherein when body warming tool is wound around the predetermined portion of the body, the warming member is pressed against the predetermined portion of the body by elastic force of the elastic member, and the density of the warming member is increased, and therefore thermal conductivity and heat insulating efficiency is enhanced.

### Effect of Invention

The body cooling tool of the present invention can be used in any portion of the body. Further, in the body cooling tool of the present invention, the water-absorbent material is employed and therefore, the cooling function can be exhibited at any time only by causing the moisture to be contained in the water-absorbent material. Further, according to the present invention, when the body cooling tool is wound around the predetermined portion of the body, the cooling member is pressed against the predetermined portion of the body by the elastic force of the elastic member, and the density of the water-absorbent material in the bag portion can be increased. Therefore, thermal conductivity of heat conducted in the water-absorbent material can be enhanced. As a result, vaporization efficiency of the moisture contained in the water-absorbent material 11 can be enhanced, and the body cooling tool with high cooling efficiency can be provided. The present invention provides the body warming tool that can realize a high heat retaining property, by the similar concept to the body cooling tool.

### BRIEF DESCRIPTION OF DRAWINGS

FIG. 1 is a schematic view illustrating one aspect of a body cooling tool according to a first embodiment of the present invention.

FIG. 2 is a schematic perspective view illustrating a cross section of when a cooling member of the body cooling tool according to the first embodiment of the present invention is cut.

FIG. 3 is a schematic view illustrating another aspect of the body cooling tool according to the first embodiment of the present invention.

FIG. 4 is a schematic view illustrating an aspect in which the length of an elastic member can be adjusted in the body cooling tool according to the first embodiment of the present invention.

FIG. 5 is a schematic view illustrating still another aspect of the body cooling tool according to the first embodiment of the present invention.

FIG. 6 is a schematic view illustrating an aspect further including an auxiliary elastic member in the body cooling tool according to the first embodiment of the present invention.

FIG. 7 is a schematic view illustrating one aspect of a cooling member that allows a bag portion to be bent in the body cooling tool according to the first embodiment of the present invention.

FIG. 8 is a schematic view illustrating another aspect of the cooling member that allows the bag portion to be bent in the body cooling tool according to the first embodiment of the present invention.

FIG. 9 is a schematic view illustrating one aspect of a body cooling tool according to a modification of the first embodiment of the present invention.

FIG. 10 is a schematic view illustrating another aspect of the body cooling tool according to the modification of the first embodiment of the present invention.

FIG. 11 is a schematic view illustrating still another aspect of the body cooling tool according to the modification of the first embodiment of the present invention.

FIG. 12(a) is a photograph illustrating an ultrafine water-absorbent polymer used in the first embodiment of the present invention, and FIG. 12(b) is a photograph illustrating a typical water-absorbent polymer.

FIG. 13 is a schematic view illustrating one aspect of a body cooling tool according to a second embodiment of the present invention.

FIG. 14 is a schematic view illustrating another aspect of the body cooling tool according to the second embodiment of the present invention.

FIG. 15 is a schematic view illustrating one aspect of a bendable cooling member (water-absorbent cloth) in the body cooling tool according to the second embodiment of the present invention.

FIG. 16 is a schematic view illustrating another aspect of a bendable cooling member (water-absorbent cloth) in the body cooling tool according to the second embodiment of the present invention.

FIG. 17 is a schematic view illustrating one aspect of a body cooling tool according to a modification of the second embodiment of the present invention.

FIG. 18 is a schematic view illustrating another aspect of the body cooling tool according to the modification of the second embodiment of the present invention.

FIG. 19 is a schematic view illustrating still another aspect of the body cooling tool according to the modification of the second embodiment of the present invention.

FIG. 20 is a schematic view illustrating one aspect of a body warming tool according to a third embodiment of the present invention.

### DESCRIPTION OF EMBODIMENTS

### First Embodiment

### <<Configuration>>

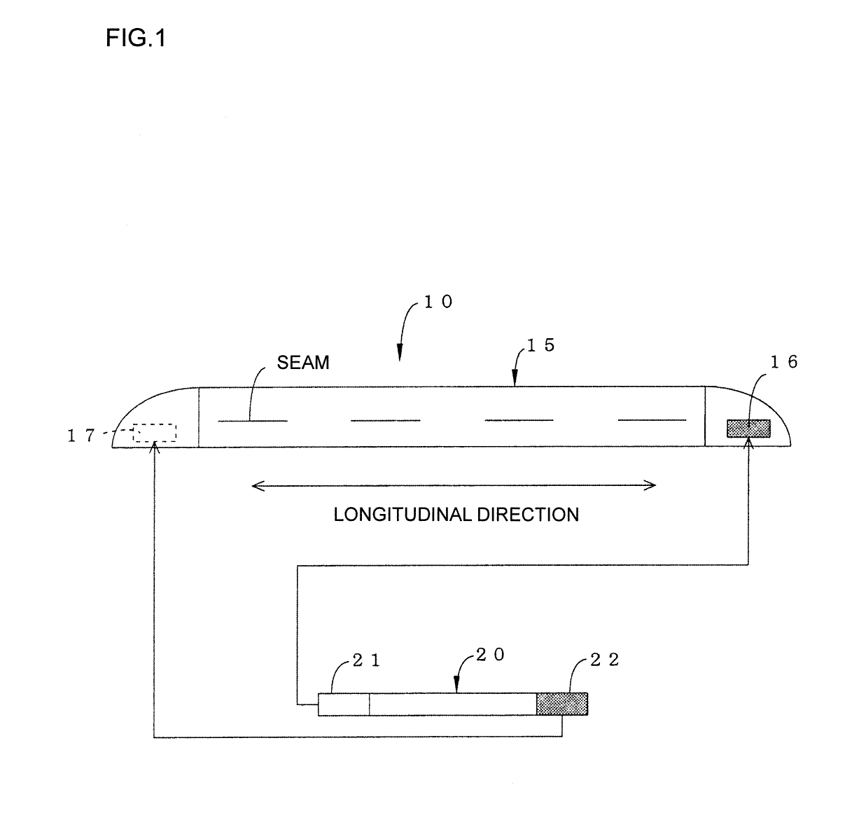

Hereinafter, a first embodiment of a body cooling tool according to the present invention will be described with reference to the drawings. Here, FIGS. 1 to 12 are diagrams for describing the first embodiment of the present invention.

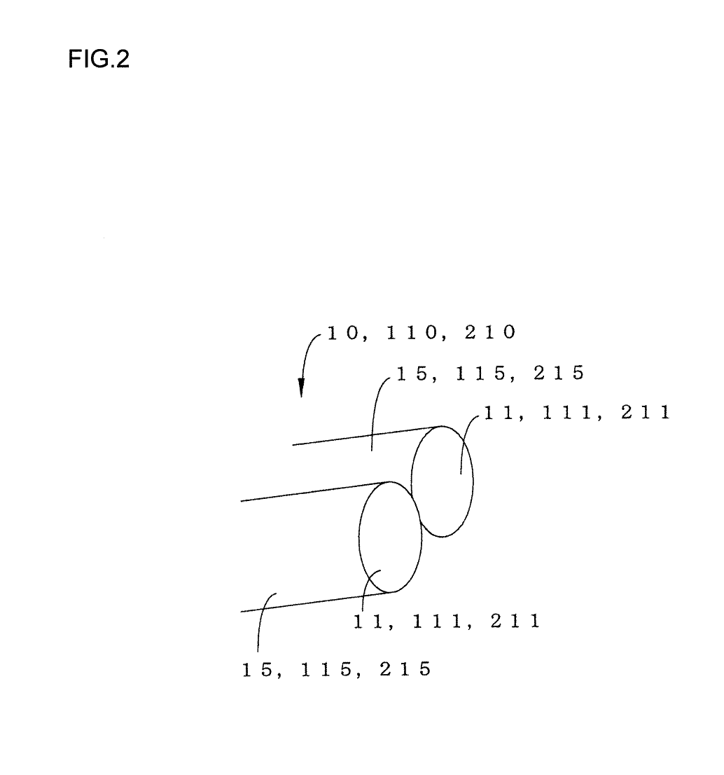

The body cooling tool of the present embodiment is wound around a predetermined portion of a body, such as a head portion, an arm portion, a leg portion, or a waist portion, and cools the portion. The body cooling tool includes, as illustrated in FIG. 1, a cooling member 10 including a water-absorbent material 11 (see FIG. 2) and a bag portion 15 being a narrow long shape and including the water-absorbent material 11 therein, and an elastic member 20 provided at an end portion of the bag portion 15. When the cooling member 10 is immersed in water, moisture infiltrating through the bag portion 15 is contained in the water-absorbent material 11. As a result, the water-absorbent material 11 is expanded. Then, the moisture contained in the water-absorbent material is vaporized and heat is drawn so that a cooling function can be realized. Note that FIG. 2 illustrates an aspect in which the water-absorbent material 11 contains the moisture and is expanded. As illustrated in FIG. 1, the bag portion 15 is sewed along a longitudinal direction (there is a seam along the longitudinal direction), and when the water-absorbent material 11 contains the moisture and is expanded, the bag portion 15 is expanded into approximately cylindrical shapes, as illustrated in FIG. 2.

Further, in the body cooling tool of the present embodiment, when the body cooling tool is wound around the predetermined portion of the body, the cooling member 10 is pressed against the predetermined portion of the body by elastic force of the elastic member 20, and the density of the water-absorbent material 11 in the bag portion 15 is increased so that cooling efficiency can be enhanced.

Examples of the water-absorbent material 11 include a water-absorbent polymer, a water-absorbent sheet, water-absorbent cloth, and water-absorbent non-woven fabric. Note that the water-absorbent material 11 is favorably the water-absorbent polymer from the viewpoint of efficiently containing the moisture and being expanded, and increasing the density of the expanded material, thereby to perform cooling.

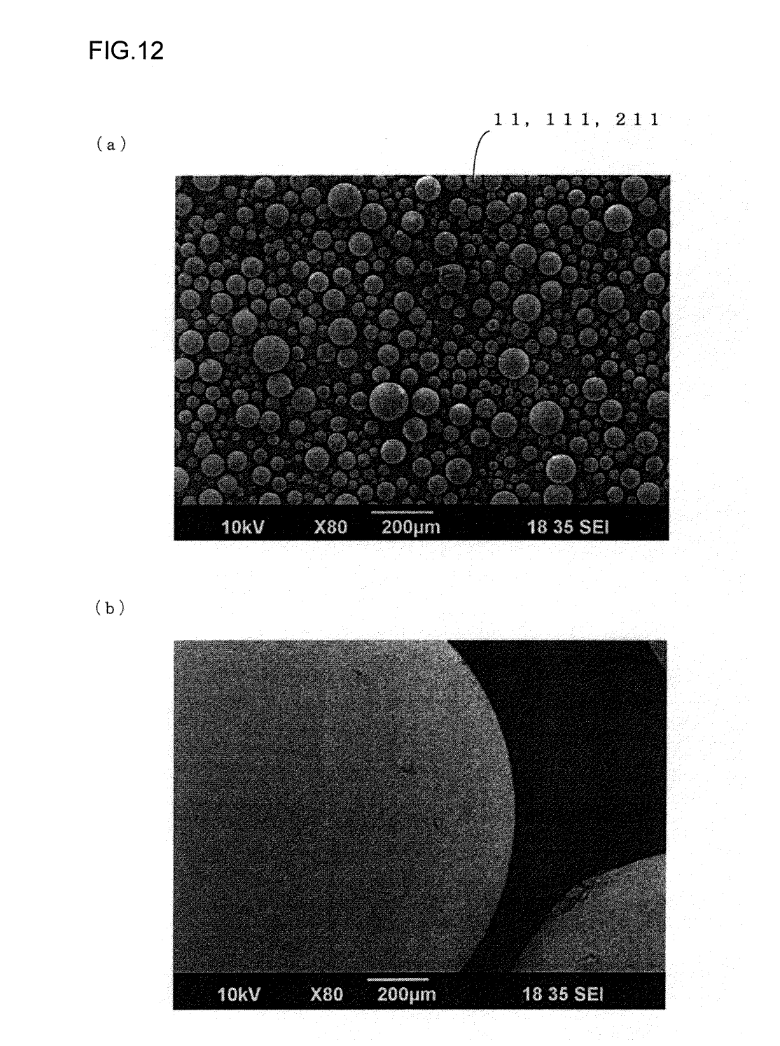

As described above, from the viewpoint of efficiently containing the moisture and being expanded, and increasing the density of the expanded material, thereby to perform cooling, an ultrafine water-absorbent polymer (water-absorbent material) 11 with a maximum diameter of 150 μm or less, favorably, 120 μm or less as illustrated in FIG. 12 (a) is beneficial among the water-absorbent polymers, unlike the typical water-absorbent polymer with the diameter of 1800 μm or more, as illustrated in FIG. 12(b).

Further, it is favorable to apply antibacterial treatment and/or deodorization treatment to the water-absorbent material 11. Further, when the water-absorbent polymer is employed as the water-absorbent material 11, it is favorable to apply the antibacterial treatment and/or deodorization treatment to each water-absorbent polymer.

Further, as described above, from the viewpoint of efficiently containing the moisture and being expanded, and increasing the density of the expanded material, thereby to perform cooling, the bag portion 15 is favorably formed of cotton. With such employment of cotton, efficiency of when the moisture is absorbed by the water-absorbent material 11 can be enhanced, and efficiency of when the moisture contained in the water-absorbent material 11 is evaporated can be enhanced. Further, it is also favorable to apply the antibacterial treatment and/or the deodorization treatment to the bag portion 15. As a material of the bag portion 15, polyester, nylon, acrylic, hemp, rayon, or a combination thereof can be used.

When cotton is employed as the material of the bag portion 15, and the ultrafine water-absorbent polymer as illustrated in FIG. 12(a) is employed as the water-absorbent material 11, the moisture can be contained in each water-absorbent polymer in about one to two minutes after the cooling member 10 is dipped in water.

Any elastic member 20 can be used as long as the cooling member 10 can be pressed against the predetermined portion of the body with the elastic force of the elastic member 20, and the density of the water-absorbent material 11 in the bag portion 15 can be increased. Examples of the elastic member 20 include a band-shaped member and a string-shaped member. Note that, from the viewpoint of pressing the cooling member 10 against the predetermined portion of the body in a well-balanced manner, and uniformly increasing the density of the water-absorbent material 11 in the bag portion 15, the elastic member 20 favorably has a certain width, and is favorably formed into a band shape with a width of 5 mm or more, more favorably formed into a band shape with a width of 10 mm or more, and still more favorably formed into a band shape with a width of 20 mm or more.

The elastic member 20 may employ an aspect of being fixed to the bag portion 15 of the cooling member 10 by sewing . However, as illustrated in FIG. 1, the elastic member 20 may employ an aspect of being freely attachable/detachable to/from the bag portion 15 of the cooling member 10. When the elastic member 20 is fixed by sewing, for example, a risk of losing any of the elastic member 20 and the cooling member 10 can be decreased, which is different from the aspect of being freely attachable/detachable to/from the bag portion 15.

When the elastic member 20 is freely attachable/detachable to/from the bag portion 15 of the cooling member 10, as illustrated in FIG. 1, a first bag attaching portion (first cooling member attaching portion) 16 may be provided at one end (an end portion on the right side in FIG. 1) of end portions of the bag portion 15, a second bag attaching portion (second cooling member attaching portion) 17 may be provided at the other end (an end portion on the left side in FIG. 1) of the end portions of the bag portion 15, and the first bag attaching portion 16 may be freely attachable/detachable to/from the second bag attaching portion 17.

Further, when the first bag attaching portion 16 is freely attachable/detachable to/from the second bag attaching portion 17, an aspect in which a first elastic attaching portion 21 is provided at one end (an end portion on the left side in FIG. 1) of end portions of the elastic member 20, a second elastic attaching portion 22 is provided at the other end (an end portion on the right side in FIG. 1) of the end portions of the elastic member 20, and the first bag attaching portion 16 is freely attachable/detachable to/from the first elastic attaching portion 21 and the second bag attaching portion 17 is freely attachable/detachable to/from the second elastic attaching portion 22 can be employed.

As an example of the first bag attaching portion 16 and the second bag attaching portion 17, one of the first bag attaching portion 16 and the second bag attaching portion 17 serves as a male of a hook-and-loop fastener, and the other of the first bag attaching portion 16 and the second bag attaching portion 17 serves as a female. Further, as an example of the first elastic attaching portion 21 and the second elastic attaching portion 22, one of the first elastic attaching portion 21 and the second elastic attaching portion 22 serves as a male of a hook-and-loop fastener, and the other of the first elastic attaching portion 21 and the second elastic attaching portion 22 serves as a female of the hook-and-loop fastener. To cause the first bag attaching portion 16 to be freely attachable/detachable to/from the first elastic attaching portion 21, and the second bag attaching portion 17 to be freely attachable/detachable to/from the second elastic attaching portion 22, the first elastic attaching portion 21 serves as the female of the hook-and-loop fastener, the second bag attaching portion 17 serves as the female of the hook-and-loop fastener, and the second elastic attaching portion 22 serves as the male of the hook-and-loop fastener, when the first bag attaching portion 16 serves as the male of the hook-and-loop fastener. On the other hand, when the first bag attaching portion 16 serves as the female of the hook-and-loop fastener, the first elastic attaching portion 21 serves as the male of the hook-and-loop fastener, the second bag attaching portion 17 serves as the male of the hook-and-loop fastener, and the second elastic attaching portion 22 serves as the female of the hook-and-loop fastener.

When the first bag attaching portion 16, the second bag attaching portion 17, the first elastic attaching portion 21, and the second elastic attaching portion 22 of FIG. 1 are the hook-and-loop fasteners and the hook-and-loop fastener provided at the first bag attaching portion 16 is positioned on a front side of a sheet surface of FIG. 1, the hook-and-loop fastener provided at the second bag attaching portion 17 is positioned on a back side of the sheet surface of FIG. 1. When the hook-and-loop fastener provided at the first elastic attaching portion 21 is positioned on the back side of the sheet surface of FIG. 1, the hook-and-loop fastener provided at the second elastic attaching portion 22 is positioned on the front side of the sheet surface of FIG. 1. Then, the hook-and-loop fastener provided at the first elastic attaching portion 21 is attached to the hook-and-loop fastener provided at the first bag attaching portion 16, and the hook-and-loop fastener provided at the second elastic attaching portion 22 is attached to the hook-and-loop fastener provided at the second bag attaching portion 17 so that the elastic member 20 can be attached to the cooling member 10.

When the elastic member 20 is freely attachable/detachable to/from the bag portion 15 of the cooling member 10, various aspects can be used in addition to the above-described hook-and-loop fasteners. For example, the elastic member 20 can be freely attachable/detachable to/from the bag portion 15 using a clip, a button, a magnet, an eye and a hook, a zipper, a buckle, or the like. More specifically, one end of the elastic member 20 and one end of the cooling member 10 can be fastened with the clip, and the other end of the elastic member 20 and the other end of the cooling member 10 can be fastened with the clip. Further, the button provided at the one end of the elastic member 20 and the button provided at the one end of the cooling member 10 can be fastened, and the button provided at the other end of the elastic member 20 and the button provided at the other end of the cooling member 10 can be fastened. Further, the magnet provided at the one end of the elastic member 20 and the magnet provided at the one end of the cooling member 10 can be fastened, and the magnet provided at the other end of the elastic member 20 and the magnet provided at the other end of the cooling member 10 can be fastened. Further, the hook or an eye provided at the one end of the elastic member 20 and the hook or the eye provided at the one end of the cooling member 10 can be fastened, and the hook or the eye provided at the other end of the elastic member 20 and the hook or the eye provided at the other end of the cooling member 10 can be fastened. Further, the one end of the elastic member 20 and the one end of the cooling member 10 can be fastened with the zipper, and the other end of the elastic member 20 and the other end of the cooling member 10 can be fastened with the zipper. Further, the buckle can be provided at one of the one end of the elastic member 20 and the one end of the cooling member 10, and the other of the one end of the elastic member 20 and the one end of the cooling member 10 can be sandwiched and fastened with the buckle, and the buckle can be provided at one of the other end of the elastic member 20 and the other end of the cooling member 10, and the other of the other end of the elastic member 20 and the other end of the cooling member 10 can be sandwiched and fastened with the buckle.

Each of the above-described hook-and-loop fastener, the clip, the button, the magnet, the eye and the hook, the zipper, and the buckle can become the attaching portion (the first bag attaching portion 16, the second bag attaching portion 17, the first elastic attaching portion 21, the second elastic attaching portion 22, or the like) . Further, how to fasten with the hook-and-loop fastener, the clip, the button, the magnet, the eye and the hook, the zipper, and the buckle can be appropriately combined. For example, the one end of the elastic member 20 and the one end of the cooling member 10 can be fastened with the clip, and the other end of the elastic member 20 and the other end of the cooling member 10 can be fastened with the zipper.

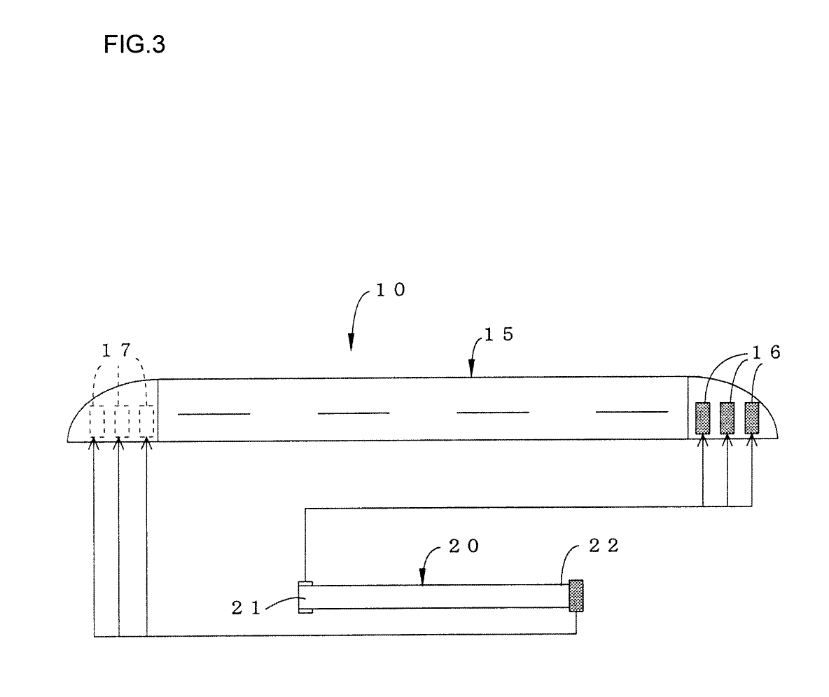

Further, attaching positions of both ends or an attaching position of one end of the elastic member 20 can be changed with respect to the cooling member 10. In the aspect illustrated in FIG. 3, a plurality of (more specifically, three) first bag attaching portions 16 are provided at the one end of the bag portion 15 of the cooling member 10, and a plurality of (more specifically, three) second bag attaching portions 17 are provided at the other end of the bag portion 15 of the cooling member 10. Then, the attaching positions of the both ends of the elastic member 20 can be changed with respect to the cooling member 10. As a specific example of such an aspect, an aspect in which a plurality of hook-and-loop fasteners is provided at each of the both ends of the one end and the other end of the bag portion 15, an aspect in which a plurality of buttons is provided at each of the both ends of the one end and the other end of the bag portion 15, an aspect in which a plurality of magnets is provided at the both ends of the one end and the other end of the bag portion 15, or the like can be employed. Note that attaching positions of the both ends or an attaching position of the one end of the cooling member 10 may be changed with respect to the elastic member 20. That is, a plurality of the first elastic attaching portions 21 and/or second elastic attaching portions 22 is provided at the both ends or the one end of the elastic member 20, and the attaching positions of the both ends or the attaching position of the one end of the cooling member 10 can be changed with respect to the elastic member 20 depending on which first elastic attaching portion 21 and/or second elastic attaching portion 22 is attached to the first bag attaching portion 16 and the second bag attaching portion 17 of the cooling member 10.

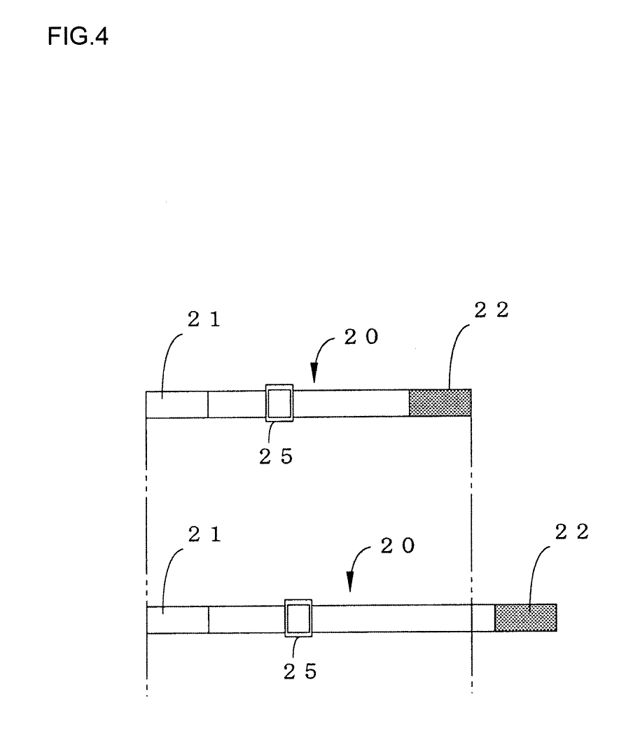

Further, as illustrated in FIG. 4, the length of the elastic member 20 maybe freely adjustable. As an example, the elastic member 20 has a length adjustment portion 25, and the length of the elastic member 20 may just be adjusted by adjusting the length of the length adjustment portion 25. Note that the upper drawing of FIG. 4 illustrates a case in which the length of the elastic member 20 is made short with the adjustment portion 25, and the lower drawing of FIG. 4 illustrates a case in which the length of the elastic member 20 is made long with the adjustment portion 25. Note that an adjustment portion for adjusting the length of the bag portion 15 may be provided at the bag portion 15 of the cooling member 10, and the length of the cooling member 10 may be freely adjustable by adjusting the length of the bag portion 15.

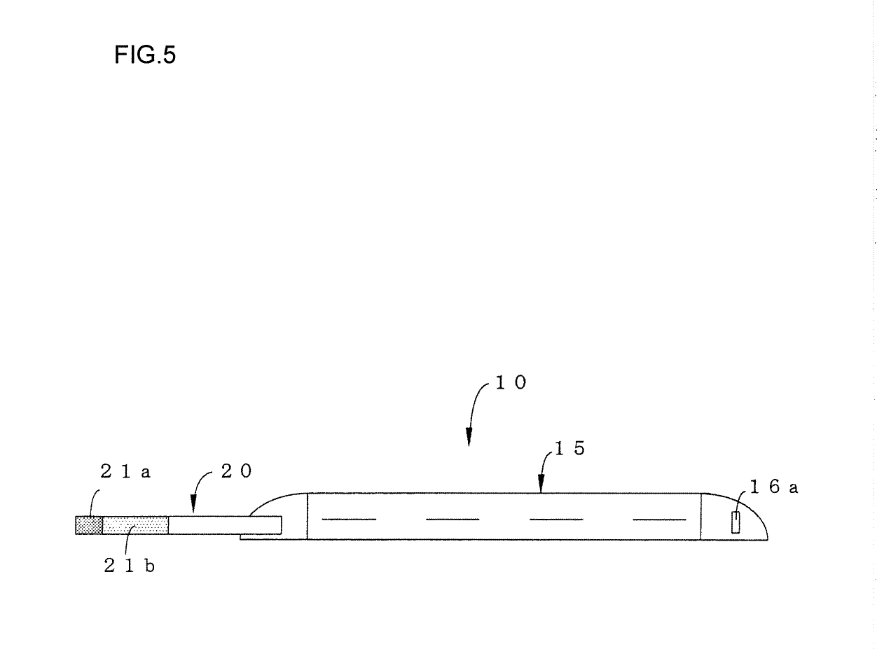

Further, a hole portion may be provided at one of the one end and the other end of the bag portion 15, and the end portion of the elastic member 20 may be attached to the other of the one end and the other end of the bag portion 15. In the aspect illustrated in FIG. 5, a hole portion 16a is provided at the one end of the bag portion 15, and the other end of the elastic member 20 is attached to the other end of the bag portion 15. Note that the other end of the bag portion 15 and the other end of the elastic member 20 may be fixed, or may be freely attachable/detachable to/from each other. In the aspect illustrated in FIG. 5, a hook-and-loop fastener 21a is provided at the one end of the elastic member 20, and the one end of the elastic member 20 is brought to pass through the hole portion 16a and is folded back so that the hook-and-loop fastener 21a may just be able to be attached to a surface 21b of the elastic member 20 after folded back. In this aspect, the attaching position of the hook-and-loop fastener 21a after passed through the hole portion 16a and folded back with respect to the surface 21b of the elastic member 20 is adjusted so that the length of the elastic member 20 can be substantially adjusted. Note that the term “the length of the elastic member is freely adjustable” described in claims includes such an aspect.

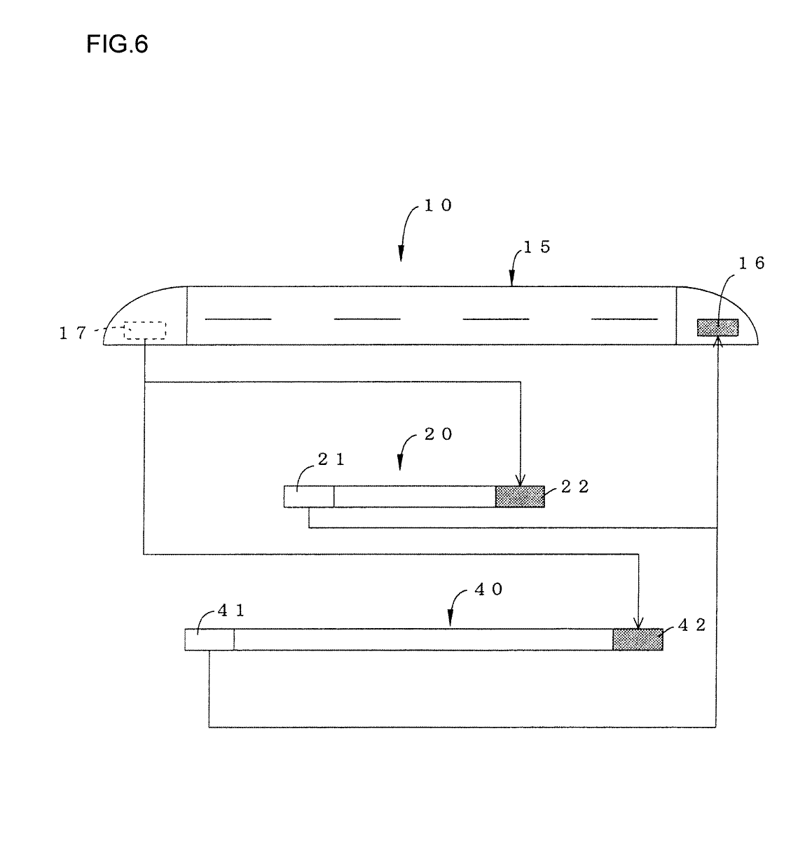

Further, as illustrated in FIG. 6, the body cooling tool of the present embodiment may further include an auxiliary elastic member 40 having a different length from the elastic member 20. The auxiliary elastic member 40 has the same configuration as the elastic member 20 except the length. Then, by use of the auxiliary elastic member 40 instead of the elastic member 20, the body cooling tool can be used in a different portion of the body. As an example, when the elastic member 20 is wound around a head portion of a human body and is relatively short, the relatively long auxiliary elastic member 40 be wound around a waist portion of a human body may be provided, or the shorter auxiliary elastic member 40 be wound around an arm of a human body may be provided. Three or more auxiliary elastic members 40 may be provided, and may just have lengths matched with various portions such as the head portion, the arm portion, the leg portion, and the waist portion. By the way, in the present application, the “body” has a concept not only including a “human body”, but also including bodies of animals other than humans. As illustrated in FIG. 6, a first auxiliary elastic attaching portion 41 corresponding to the first elastic attaching portion 21 is provided at one end of an end portion of the auxiliary elastic member 40, and a second auxiliary elastic attaching portion 42 corresponding to the second elastic attaching portion 22 is provided at the other end of an end portion of the auxiliary elastic member 40.

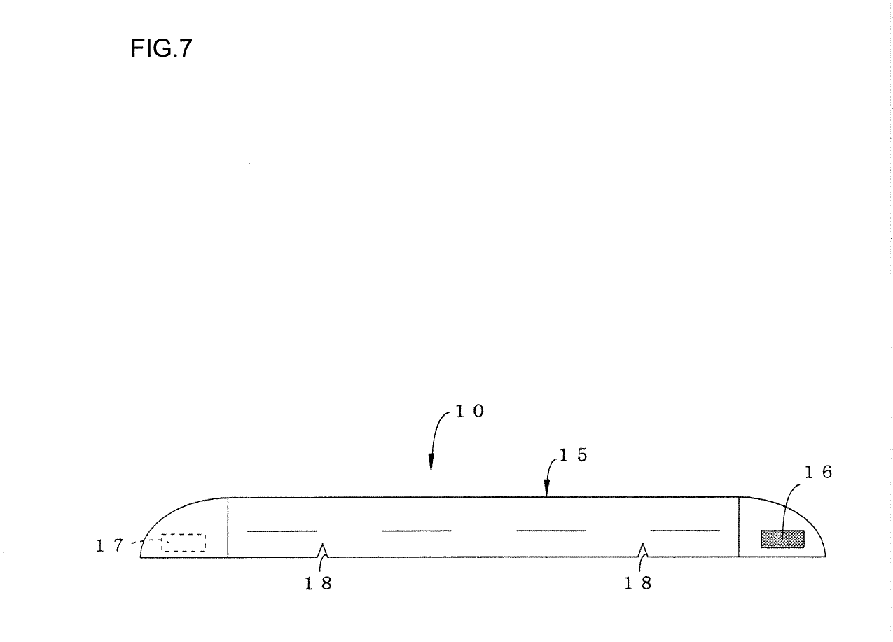

Further, as illustrated in FIG. 7, notches 18 are provided at predetermined portions of the bag portion 15 of the present embodiment, and the bag portion 15 may be able to be bent with the notches 18 in a direction perpendicular to the longitudinal direction of the bag portion 15. In this aspect, the cooling member 10 may cool at least the back side of the neck of the human body, and may be able to pass above ears of the human body. This aspect is mainly used to cool the back side of the neck of the human body. From the viewpoint of symmetrically bending the bag portion 15, the two notches 18 are favorably provided at positions symmetrical to a surface perpendicular to the longitudinal direction of the bag portion 15 and passing through the center of the bag portion 15.

Bag coupling tools (coupling tools) 19a and 19b may be provided at predetermined portions of the bag portion 15, instead of or in addition to the notches 18, and two portions of the bag portion 15 are coupled with the bag coupling tools 19a and 19b so that the bag portion 15 may be able to be bent in the direction perpendicular to the longitudinal direction of the bag portion 15. In this aspect, the cooling member 10 may cool at least the back side of the neck of the human body, and may be able to pass above the ears of the human body. This aspect is also used to cool the back side of the neck of the human body. Note that the bag coupling tools 19a and 19b are, for example, the buttons, magnets, hook-and-loop fasteners, or zippers, and the bag coupling tool 19a is coupled with the bag coupling tool 19b so that the bag portion 15 can be bent in the direction perpendicular to the longitudinal direction of the bag portion 15. Further, even in this aspect, from the viewpoint of symmetrically bending the bag portion 15, the two bag coupling tools 19a and 19b are favorably provided at positions symmetrical to the surface perpendicular to the longitudinal direction of the bag portion 15 and passing through the center of the bag portion 15.

By the way, when the hook-and-loop fasteners are employed as the attaching portions (the first bag attaching portion 16, the second bag attaching portion 17, the first elastic attaching portion 21, the second elastic attaching portion 22, and the like), the hook-and-loop fasteners can be easily detached with one hand. When the clips are employed as the attaching portions (the first bag attaching portion 16, the second bag attaching portion 17, the first elastic attaching portion 21, the second elastic attaching portion 22, and the like), the attaching position of the elastic member 20 with respect to the bag portion 15 can be appropriately adjusted by changing portions clipped with the clips, and the clips can be easily detached with one hand. When the buttons are employed as the attaching portions (the first bag attaching portion 16, the second bag attaching portion 17, the first elastic attaching portion 21, the second elastic attaching portion 22, and the like), the attaching portions can be firmly attached with the buttons. Further, when the magnets are employed as the attaching portions (the first bag attaching portion 16, the second bag attaching portion 17, the first elastic attaching portion 21, the second elastic attaching portion 22, and the like), the magnets can be easily attached by being brought close to each other, and can be easily detached with one hand. Further, when combinations of the eyes and the hooks are employed as the attaching portions (the first bag attaching portion 16, the second bag attaching portion 17, the first elastic attaching portion 21, the second elastic attaching portion 22, and the like), the eyes and the hooks can be easily detached with one hand. Further, when the zippers are employed as the attaching portions (the first bag attaching portion 16, the second bag attaching portion 17, the first elastic attaching portion 21, the second elastic attaching portion 22, and the like) , the zippers can be firmly attached, and the shape can be made unique and excellent in design. Further, when the buckles are employed as the attaching portions (the first bag attaching portion 16, the second bag attaching portion 17, the first elastic attaching portion 21, the second elastic attaching portion 22, and the like), the buckles can be firmly attached.

### <<Effects>>

Next, effects achieved by the present embodiment made of the above-described configurations, and effects that have not yet been described or are especially important will be described.

The body cooling tool of the present embodiment can be used in any portion of the body. Further, in the body cooling tool of the present embodiment, the water-absorbent material 11 is employed and therefore, the cooling function can be exhibited at any time only by causing the moisture to be contained in the water-absorbent material 11.

Further, according to the present embodiment, when the body cooling tool is wound around the predetermined portion of the body, the cooling member 10 is pressed against the predetermined portion of the body by the elastic force of the elastic member 20, and the density of the water-absorbent material 11 in the bag portion 15 can be increased. Therefore, thermal conductivity of heat conducted in the water-absorbent material 11 in the bag portion 15 can be enhanced. As a result, vaporization efficiency of the moisture contained in the water-absorbent material 11 can be enhanced, and the body cooling tool with high cooling efficiency can be provided.

Further, in the present embodiment, the body cooling tool includes the water-absorbent material 11. Then, the water-absorbent material 11 absorbs the moisture infiltrated through the bag portion 15 and is expanded, and has soft texture . Therefore, even if the body cooling tool is tightened with the elastic member 20, a user is less likely to have an uncomfortable feeling in the tightened portion. Even if the cooling efficiency is enhanced by tightening the body cooling tool and increasing the density of the water-absorbent material 11 in the bag portion 15, the user cannot have an uncomfortable feeling. This point is beneficial.

In the present embodiment, when the water-absorbent material 11 contains the moisture and is expanded, the water-absorbent material 11 is formed into cylindrical shapes (including approximately cylindrical shapes), as illustrated in FIG. 2. More specifically, as illustrated in FIG. 2, the water-absorbent material 11 is expanded as two cylindrical shapes (including approximately cylindrical shapes), and these two cylindrical-shaped water-absorbent materials 11 come in contact with the body. The water-absorbent materials 11 become the cylindrical shapes as described above so that even if the body cooling tool is tightened, the uncomfortable feeling experienced by the user who wears the body cooling tool of the present embodiment can be decreased. Further, a plurality of (two in the present embodiment) cylindrical shapes are formed, instead of one so that a thickness (a thickness in a direction perpendicular to a surface including the longitudinal direction and a short direction of the bag portion 15) can be made adequate and the vaporization of the moisture can be efficiently caused while an area where the user and the body cooling tool come in contact with each other is increased, whereby high cooling efficiency can be realized.

In the present embodiment, when the water-absorbent polymer is employed as the water-absorbent material 11, the density can be more uniformly increased when the body cooling tool is tightened with the elastic member 20, and the cooling efficiency can be enhanced. Especially, in the case of the ultrafine water-absorbent polymer (water-absorbent material) 11 with the maximum diameter of 150 μm or less, favorably, 120 μm or less (see FIG. 12(a)), the density can be further increased when the body cooling tool is tightened, and it is thus favorable . As described above, when the water-absorbent polymer is employed as the water-absorbent material 11, the water-absorbent polymers 11 illustrated in FIG. 12(a) are condensed and the density is increased when the body cooling tool is tightened with the elastic member 20.

Further, when the elastic member 20 has the band shape, especially, the band shape with a width of 5 mm or more, more favorably the band shape with a width of 10 mm or more, or still more favorably the band shape with a width of 20 mm or more, the cooling member 10 is pressed against the predetermined portion of the body in a well-balanced manner, and the density of the water-absorbent material 11 in the bag portion 15 can be uniformly increased. Therefore, it is favorable.

Further, when the aspect in which the elastic member 20 is freely attachable/detachable to/from the bag portion 15 of the cooling member 10 is employed, use of the cooling member 10 only, and use of an aspect in which the elastic member 20 is attached to the cooling member 10 can be appropriately selected. Further, by adding predetermined pulling force, an aspect in which the elastic member 20 is freely detachable from the bag portion 15 of the cooling member 10 can be employed. In this case, the elastic member 20 is removed from the bag portion 15 of the cooling member 10 when the predetermined pulling force is added. Therefore, safety can be enhanced. To be specific, when the body cooling tool according to the present embodiment is used in a factory, for example, a possibility of the body cooling tool of the present embodiment getting entangled with a machine in the factory is not “0”. If the elastic member 20 can be removed from the bag portion 15 of the cooling member 10 when the predetermined pulling force is added, when such an accident occurs, the elastic member 20 and the cooling member 10 of the body cooling tool that gets caught in the machine naturally come off. Therefore, the user can be prevented from getting caught in the machine. Examples of the attaching portions (the first bag attaching portion 16, the second bag attaching portion 17, the first elastic attaching portion 21, the second elastic attaching portion 22, and the like) used in such aspect include the hook-and-loop fastener and the magnet.

Further, when the elastic member 20 is freely attachable/detachable to/from the bag portion 15 of the cooling member 10, the first bag attaching portion 16 is provided at the one end of the end portion of the bag portion 15 and the second bag attaching portion 17 is provided at the other end of the end portion of the bag portion 15, and the first bag attaching portion 16 is freely attachable/detachable to/from the second bag attaching portion 17, winding only the cooling member 10 around another portion of the body can be performed, in addition to winding the cooling member 10 around the predetermined portion of the body while tightening the body cooling tool with the elastic member 20. Therefore, the use of the body cooling tool of the present embodiment can be broadened, and can meet the wide need of the user.

Further, when the aspect in which the attaching positions of the both ends or the attaching position of one end of the elastic member 20 can be changed with respect to the cooling member 10 is employed (see FIG. 3), the degree of tightening of when the elastic member 20 is wound can be adjusted, or the attaching position can be changed in accordance with the size of the body of the user. Therefore, the aspect can satisfy the user's requirements and is beneficial.

Further, when the aspect in which the length of the elastic member 20 is freely adjustable is employed (see FIG. 4), the degree of tightening of when the elastic member 20 is wound can be adjusted, or the attaching position can be changed in accordance with the size of the body of the user. Therefore, the aspect can satisfy the user's requirements and is beneficial.

Further, when the aspect in which the auxiliary elastic member 40 having a different length from the elastic member 20 is further included is employed (see FIG. 6), the body cooling tool can be used in a different portion of the body. Therefore, the use of the body cooling tool of the present embodiment can be broadened, and can meet the wide need of the user.

When the aspect in which the notches 18 are provided at the predetermined portions of the bag portion 15 of the present embodiment, and the bag portion 15 can be bent in the direction perpendicular to the longitudinal direction of the bag portion 15 with the notches 18 is employed (see FIG. 7), the usage of cooling the back side of the neck of the human body can be employed without providing an uncomfortable feeling, in addition to the usage of cooling the head portion of the human body. That is, if the aspect in which the bag portion 15 can be bent in the direction perpendicular to the longitudinal direction of the bag portion 15 with the notches 18 is not employed, the bag portion 15 gets caught on the ears when the bag portion 15 is brought to the back side of the neck of the human body. Therefore, the position of the bag portion 15 needs to be shifted above the ears (otherwise the ears are blocked and sounds cannot be heard). If the position of the bag portion 15 is shifted above the ears as described above, the back side of the neck, which should be most intensively cooled so that the user can feel good, cannot be cooled. From this aspect, by employing the aspect in which the bag portion 15 is bent in the direction perpendicular to the longitudinal direction of the bag portion 15 with the notches 18, the bag portion 15 that includes the water-absorbent material 11 therein can be brought in contact with a lower side of a nape that is most intensively cooled so that the user can feel good, while the bag portion 15 is brought to pass above the ears of the human body, and the back side of the neck can be cooled.

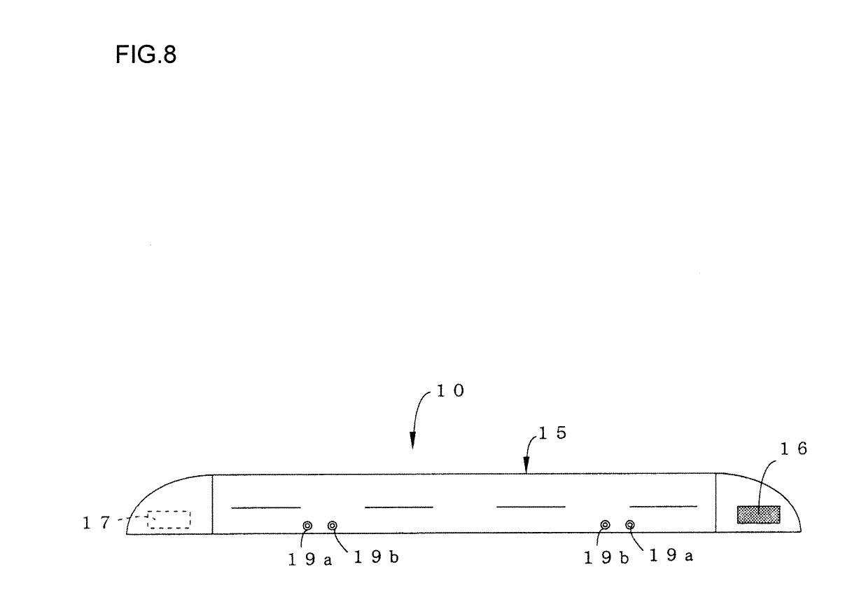

Further, as illustrated in FIG. 8, the bag coupling tools 19a and 19b are provided at the predetermined portions of the bag portion 15, instead of or in addition to the notches 18 provided at the predetermined portions of the bag portion 15, and the two places of the bag portion 15 are coupled with the bag coupling tools 19a and 19b so that the bag portion 15 may be bent in the direction perpendicular to the longitudinal direction of the bag portion 15. Even when this aspect is employed, the bag portion 15 that includes the water-absorbent material 11 therein can be brought in contact with the lower side of the neck that is most intensively cooled so that the user can feel good, while the bag portion 15 is brought to pass above the ears of the human body, and the back side of the neck can be cooled.

### <Modification>

Unlike the above-described aspects, two or more cooling members 10 may be provided. Description will be given using an aspect in which two cooling members 10 are provided as an example. Note that the same portion as the above-described embodiment is denoted with the same reference sign, a corresponding portion is denoted with a numerical number obtained by adding “100” or “200”, and detailed description is omitted, in this modification.

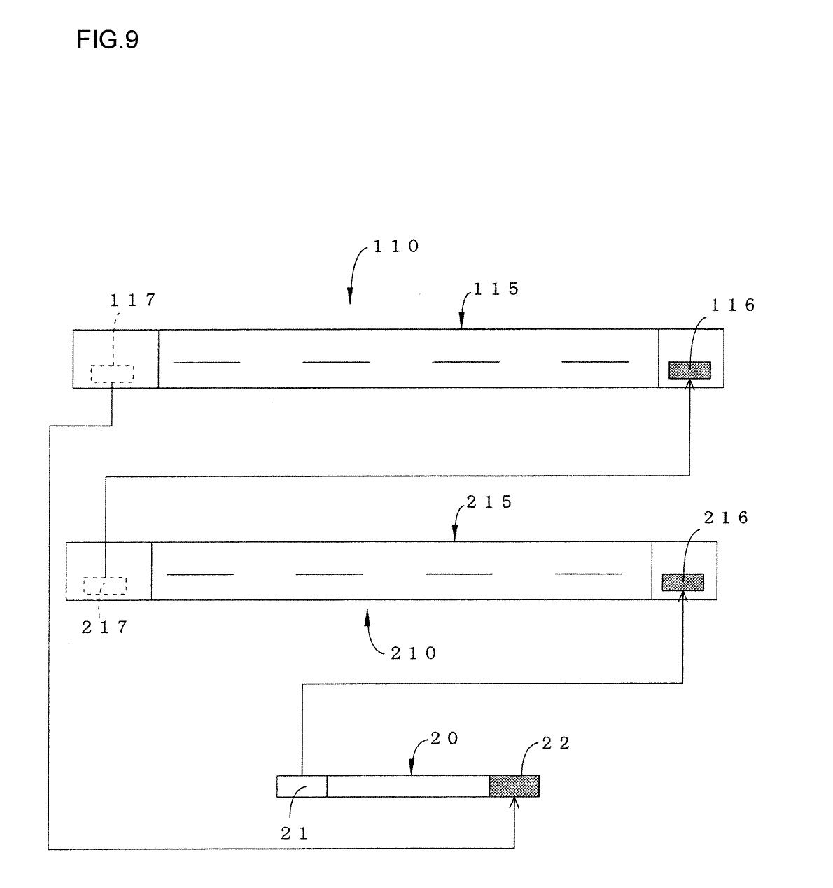

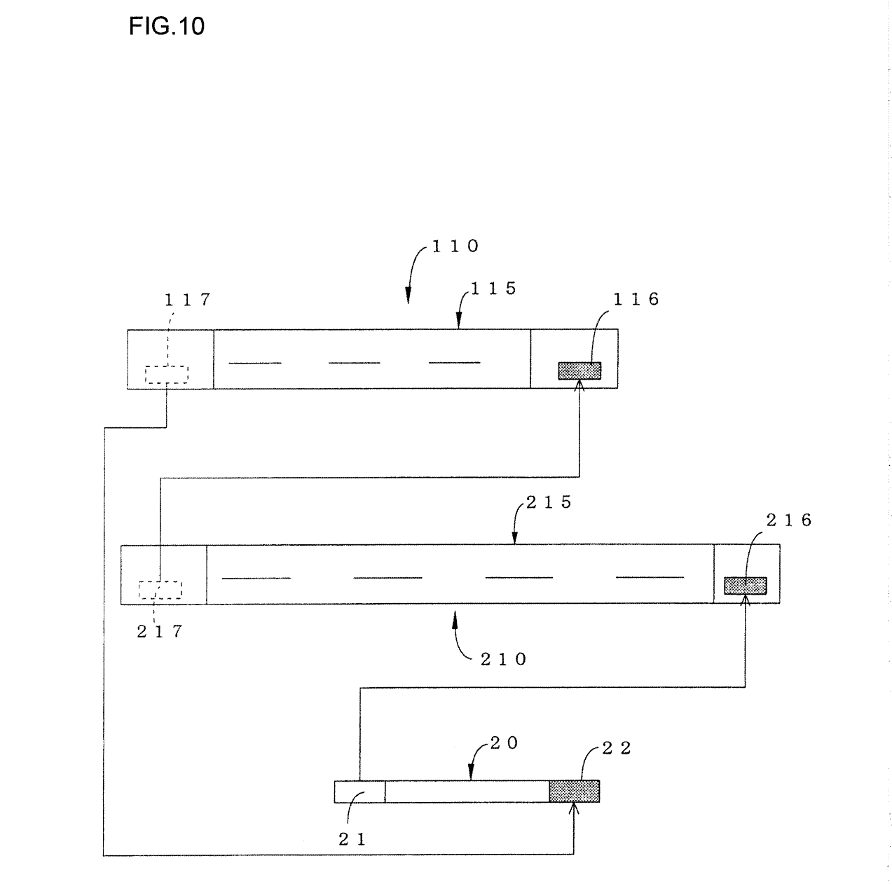

A body cooling tool in this modification includes, as illustrated in FIGS. 9 and 10, a first cooling member 110 including a first water-absorbent material 111 (see FIG. 2) and a first bag portion 115 being a narrow long shape and including the first water-absorbent material 111 therein, a second cooling member 210 including a second water-absorbent material 211 (see FIG. 2) and a second bag portion 215 being a narrow long shape and including the second water-absorbent material 211 therein, and an elastic member 20 freely attachable/detachable to/from both ends of the first bag portion 115 of the first cooling member 110 and freely attachable/detachable to/from both ends of the second bag portion 215 of the second cooling member 210. Note that, as illustrated in FIG. 9, the length of the first bag portion 115 and the length of the second bag portion 215 may be the same. However, as illustrated in FIG. 10, the length of the first bag portion 115 and the length of the second bag portion 215 may be different.

Then, moisture is contained in the first water-absorbent material 111 and the second water-absorbent material 211 so that the first water-absorbent material 111 and the second water-absorbent material 211 are expanded into cylindrical shapes, and a cooling function is exhibited by heat drawn by the moisture contained in the first water-absorbent material 111 and the second water-absorbent material 211 when vaporized.

Note that both end portions of the first bag portion 115 of the first cooling member 110 are freely attachable/detachable to/from both end portions of the second bag portion 215 of the second cooling member 210.

According to the present modification, any of following types of usage can be selected:

(1) usage of attaching portions 21 and 22 of both ends of the elastic member 20 to attaching portions 116 and 117 of both ends of the first cooling member 110;
(2) usage of the attaching the attaching portions 21 and 22 of the both ends of the elastic member 20 to attaching portions 216 and 217 of both ends of the second cooling member 210;
(3) usage of attaching the attaching portions 216 and 217 of the both ends of the second cooling member 210 to the attaching portions 116 and 117 of the both ends of the first cooling member 110; and
(4) usage of attaching the attaching portion 116 of the one end of the first cooling member 110 to the attaching portion 217 of the other end of the second cooling member 210, attaching the attaching portion 117 of the other end of the first cooling member 110 to the attaching portion 22 of the other end of the elastic member 20, and attaching the attaching portion 216 of the one end of the second cooling member 210 to the attaching portion 21 of the one end of the elastic member 20 (see FIGS. 9 and 10).

Then, in the above-described (1), (2), and (4) usage, the first cooling member 110, the second cooling member 210, or both the first cooling member 110 and the second cooling member 210 are pressed against a predetermined portion of a body by elastic force of the elastic member 20 when the body cooling tool is wound around the predetermined portion of the body.

More specifically, when the (1) usage of attaching the both ends of the elastic member 20 to the both ends of the first cooling member 110 is employed, the first cooling member 110 is pressed against the predetermined portion of the body by the elastic force of the elastic member 20 when first cooling member 110 is wound around the predetermined portion of the body. When the (2) usage of attaching the both ends of the elastic member 20 to the both ends of the second cooling member 210 is employed, the second cooling member 210 is pressed against the predetermined portion of the body by the elastic force of the elastic member 20 when the second cooling member 210 is wound around the predetermined portion of the body. When the (4) usage of attaching the attaching portion 116 of the one end of the first cooling member 110 to the attaching portion 217 of the other end of the second cooling member 210, attaching the attaching portion 117 of the other end of the first cooling member 110 to the attaching portion 22 of the other end of the elastic member 20, and attaching the attaching portion 216 of the one end of the second cooling member 210 to the attaching portion 21 of the one end of the elastic member 20 (see FIGS. 9 and 10) is employed, both the first cooling member 110 and the second cooling member 210 are pressed against the predetermined portion of the body when the first cooling member 110 and the second cooling member 210 are wound around the predetermined portion of the body.

Then, the first cooling member 110, the second cooling member 210, or both the first cooling member 110 and the second cooling member 210 are pressed against the predetermined portion of the body by the elastic force of the elastic member 20 when the body cooling tool is wound around the predetermined portion of the body so that the density of the water-absorbent materials 111 and 211 in the bag portions 115 and 215 can be increased, and as a result, cooling efficiency can be enhanced.

According to the present modification, as described above, the four types of usage can be employed:

(1) usage of attaching the both ends of the elastic member 20 to the both ends of the first cooling member 110;
(2) usage of attaching the both ends of the elastic member 20 to the both ends of the second cooling member 210;
(3) usage of attaching the attaching portions 216 and 217 of the both ends of the second cooling member 210 to the attaching portions 116 and 117 of the both ends of the first cooling member 110; and
(4) usage of attaching the attaching portion 116 of the one end of the first cooling member 110 to the attaching portion 217 of the other end of the second cooling member 210, attaching the attaching portion 117 of the other end of the first cooling member 110 to the attaching portion 22 of the other end of the elastic member 20, and attaching the attaching portion 216 of the one end of the second cooling member 210 to the attaching portion 21 of the one end of the elastic member 20 (see FIGS. 9 and 10).

Therefore, for example, when the (1) usage of attaching the both ends of the elastic member 20 to the both ends of the first cooling member 110, and the (2) usage of attaching the both ends of the elastic member 20 to the both ends of the second cooling member 210 are employed, the body cooling tool can be used by being wound around a head portion of the body. When the (4) usage of attaching the attaching portion 116 of the one end of the first cooling member 110 to the attaching portion 217 of the other end of the second cooling member 210, attaching the attaching portion 117 of the other end of the first cooling member 110 to the attaching portion 22 of the other end of the elastic member 20, and attaching the attaching portion 216 of the one end of the second cooling member 210 to the attaching portion 21 of the one end of the elastic member 20 (see FIGS. 9 and 10) is employed, the body cooling portion can be used by being wound around a waist portion or a stomach portion of the body.

When the lengths of the first bag portion 115 and the second bag portion 215 are different, as illustrated in FIG. 10, the case of employing the (1) usage of attaching the both ends of the elastic member 20 to the both ends of the first cooling member 110 and the case of employing the (2) usage of attaching the both ends of the elastic member 20 to the both ends of the second cooling member 210 can cool different portions of the body. Therefore, the use can be further broadened, and this point is beneficial.

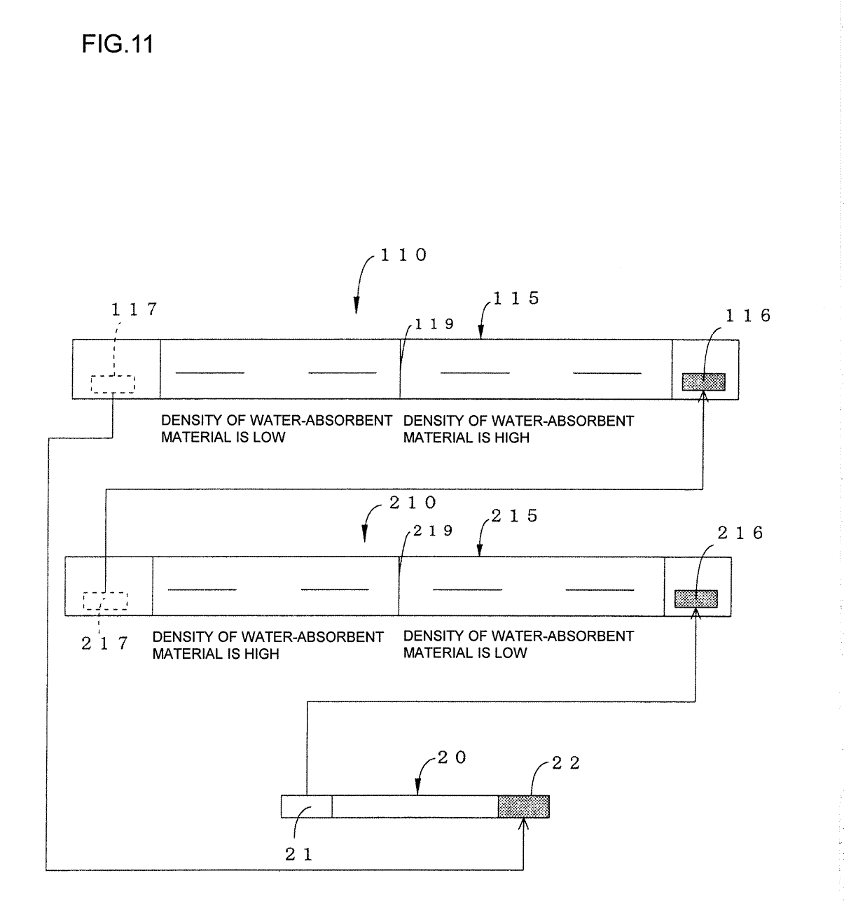

Further, in this modification, the density of the first water-absorbent material 11 included in the bag portion 115 of the first cooling member 110 and/or the density of the second water-absorbent material 211 included in the bag portion 215 of the second cooling member 210 may not be uniform and may be biased. As an example, as illustrated in FIG. 11, the density of the first water-absorbent material 111 included in the bag portion 115 of the first cooling member 110 is higher in the one end side of the first cooling member 110 than the other end side, and the density of the second water-absorbent material 211 included in the bag portion 215 of the second cooling member 210 is higher in the other end side of the second cooling member 210 than the one end side. In the aspect illustrated in FIG. 11, an approximate center of the bag portion 115 is sewed along a short direction (there is a seam 119) so that the first water-absorbent material 111 at the one end side of the first cooling member 110 and the first water-absorbent material 111 at the other end side of the first cooling member 110 are not merged. Similarly, an approximate center of the bag portion 215 is sewed along the short direction (there is a seam 219) so that the second water-absorbent material 211 at the one end side of the second cooling member 210 and the second water-absorbent material 211 at the other end side of the second cooling member 210 are not merged.

According to such an aspect, in the usage of attaching the attaching portion 116 of the one end of the first cooling member 110 to the attaching portion 217 of the other end of the second cooling member 210, attaching the attaching portion 117 of the other end of the first cooling member 110 to the attaching portion 22 of the other end of the elastic member 20, and attaching the attaching portion 216 of the one end of the second cooling member 210 to the attaching portion 21 of the one end of the elastic member 20 (see FIGS. 9 and 10), the density of the water-absorbent materials 111 and 211 can be increased in an end portion at an opposite side to the elastic member 20. More specifically, the density of the first water-absorbent material 111 of the first cooling member 110 in the end portion at the opposite side to the elastic member 20 (the end portion at the attaching portion 116 side) can be made higher than the density of the first water-absorbent material 111 of the first cooling member 110 in the end portion at the elastic member 20 side (the end portion at the attaching portion 117 side), and the density of the second water-absorbent material 211 of the second cooling member 210 in the end portion at the opposite side to the elastic member 20 (the end portion of the attaching portion 217 side) can be made higher than the density of the second water-absorbent material 211 of the second cooling member 210 in the end portion at the elastic member 20 side (the end portion at the attaching portion 216 side).

Therefore, when such an aspect is employed, when the (4) usage of attaching the attaching portion 116 of the one end of the first cooling member 110 to the attaching portion 217 of the other end of the second cooling member 210, attaching the attaching portion 117 of the other end of the first cooling member 110 to the attaching portion 22 of the other end of the elastic member 20, and attaching the attaching portion 216 of the one end of the second cooling member 210 to the attaching portion 21 of the one end of the elastic member 20 (see FIGS. 9 and 10) is employed, a portion to be cooled (the waist portion or the like) can be efficiently cooled in a focused manner.

### Second Embodiment

Next, a second embodiment of the present invention will be described.

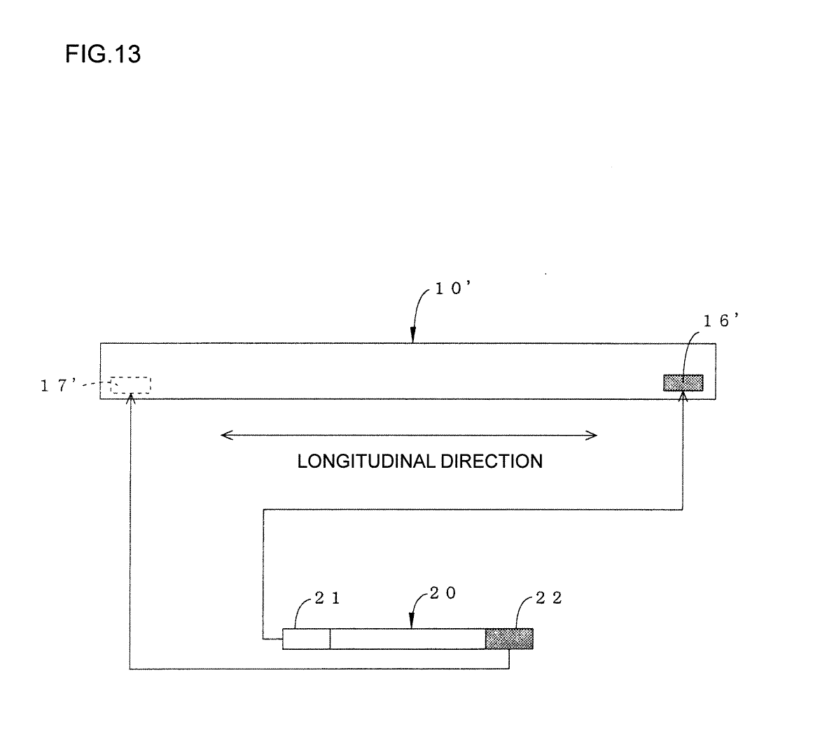

A cooling member 10 of the present embodiment is made of water-absorbent cloth, and does not include a bag portion 15 (see FIG. 13). Configurations other than the cooling member 10 being made of the water-absorbent cloth and not including the bag portion 15 are similar to the first embodiment. In the second embodiment, the same portion as the first embodiment is denoted with the same reference sign, a portion corresponding to the portion disclosed in the first embodiment is denoted with a similar reference sign (an apostrophe “'” is attached to an appropriate number), and detailed description is omitted.

According to the present embodiment, when a body cooling tool is wound around a predetermined portion of a body, the water-absorbent cloth itself can be pressed against the predetermined portion of the body without through the bag portion 15 by elastic force of an elastic member 20, and the density of a water-absorbent material can be increased and the water-absorbent material can directly adhere to the body. Therefore, heat can be directly absorbed from the body, vaporization efficiency of moisture contained in the water-absorbent cloth can be enhanced, and a body cooling tool having high cooling efficiency can be provided.

Note that the present embodiment includes an aspect that employs a “cooling member 10′” (made of the water-absorbent cloth) in place of a “cooling member 10” in all the aspects disclosed in the first embodiment. Although detailed description is not given, following aspects are included in the present embodiment. Note that, hereinafter, description will be given using a term “water-absorbent cloth 10′”, instead of “cooling member 10′”.

The present embodiment includes, as illustrated in FIG. 13, an aspect in which a first water-absorbent cloth-side attaching portion 16′ is provided at one end of an end portion of a water-absorbent cloth 10′, a second water-absorbent cloth-side attaching portion 17′ is provided at the other end of an end portion of the water-absorbent cloth 10′ , a first elastic attaching portion 21 is provided at one end of an end portion of the elastic member 20, and a second elastic attaching portion 22 is provided at the other end of an end portion of the elastic member 20, the first water-absorbent cloth-side attaching portion 16′ is freely attachable/detachable to/from the first elastic attaching portion 21 and the second water-absorbent cloth-side attaching portion 17′ is freely attachable/detachable to/from the second elastic attaching portion 22, and the first water-absorbent cloth-side attaching portion 16′ is also freely attachable/detachable to/from the second water-absorbent cloth-side attaching portion 17′.

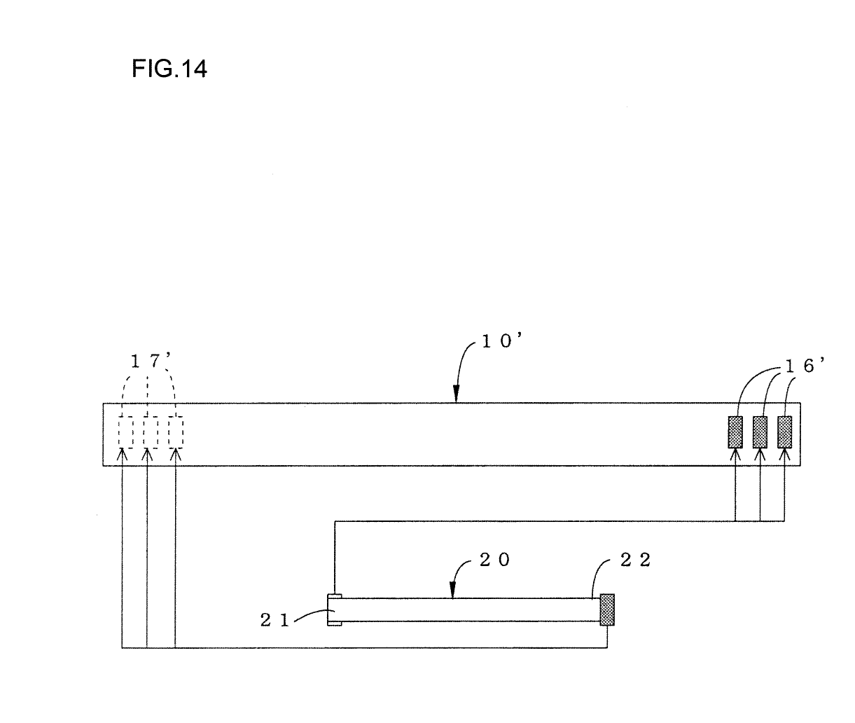

The present embodiment includes an aspect in which the elastic member 20 is formed into a band shape with a width of 5 mm or more. The present embodiment includes an aspect in which the elastic member 20 is freely attachable/detachable to/from the water-absorbent cloth 10′. The present embodiment includes an aspect in which an attaching position of the elastic member 20 with respect to the water-absorbent cloth 10′ can be changed (see FIG. 14). The present embodiment includes an aspect in which the length of the elastic member 20 is freely adjustable (see FIG. 4).

The present embodiment includes an aspect in which an auxiliary elastic member 40 having a different length from the elastic member 20 is further included (see FIG. 6).

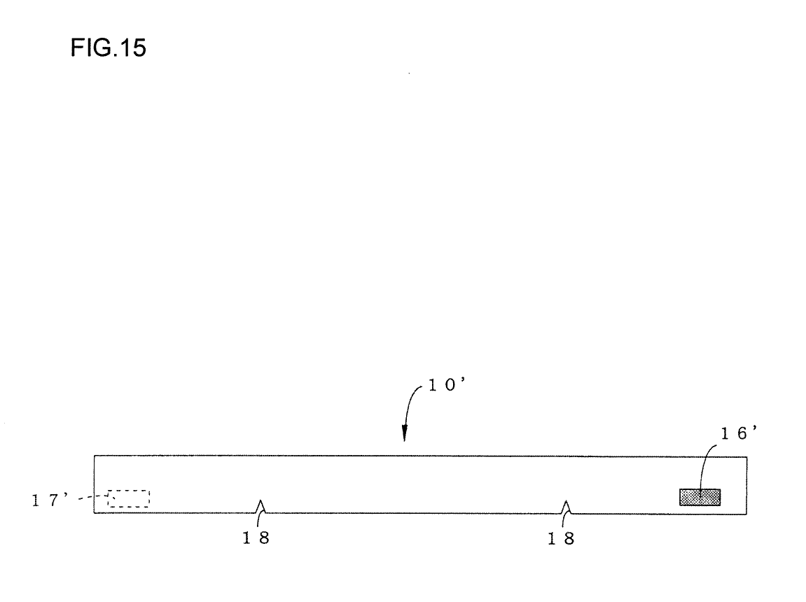

The present embodiment includes, as illustrated in FIG. 15, an aspect in which notches 18 are provided at predetermined portions of the water-absorbent cloth 10′ and the water-absorbent cloth 10′ can be bent in a direction perpendicular to a longitudinal direction of the water-absorbent cloth 10′ with the notches 18, and the water-absorbent cloth 10′ cools at least a back side of a neck of a human body and can pass above ears of the human body.

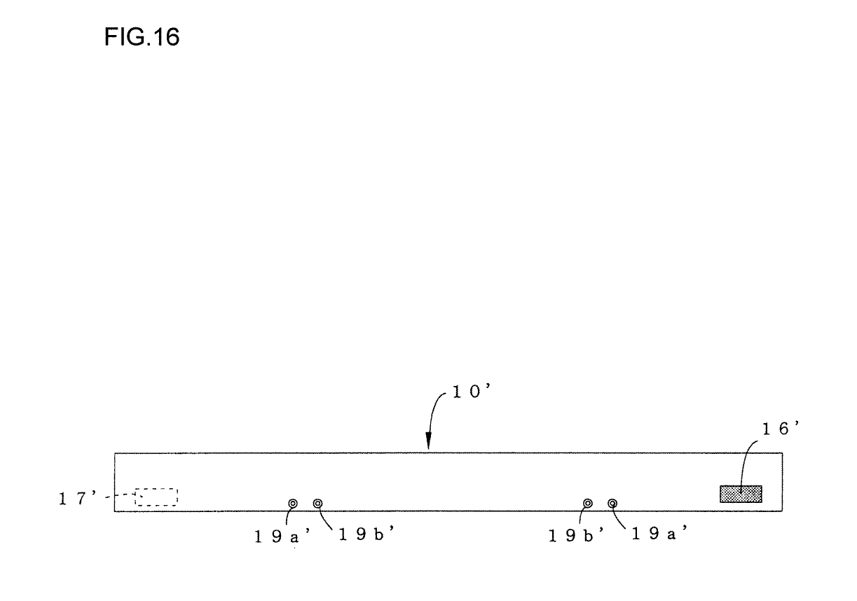

Further, the present embodiment includes, as illustrated in FIG. 16, coupling tools 19a′ and 19b′ are provided at predetermined portions of the water-absorbent cloth 10′, two places of the water-absorbent cloth 10′ are coupled with the coupling tools 19a′ and 19b′ so that the water-absorbent cloth 10′ can be bent in the direction perpendicular to the longitudinal direction of the water-absorbent cloth 10′, and the water-absorbent cloth 10′ cools at least the back side of the neck of the human body and can pass above the ears of the human body.

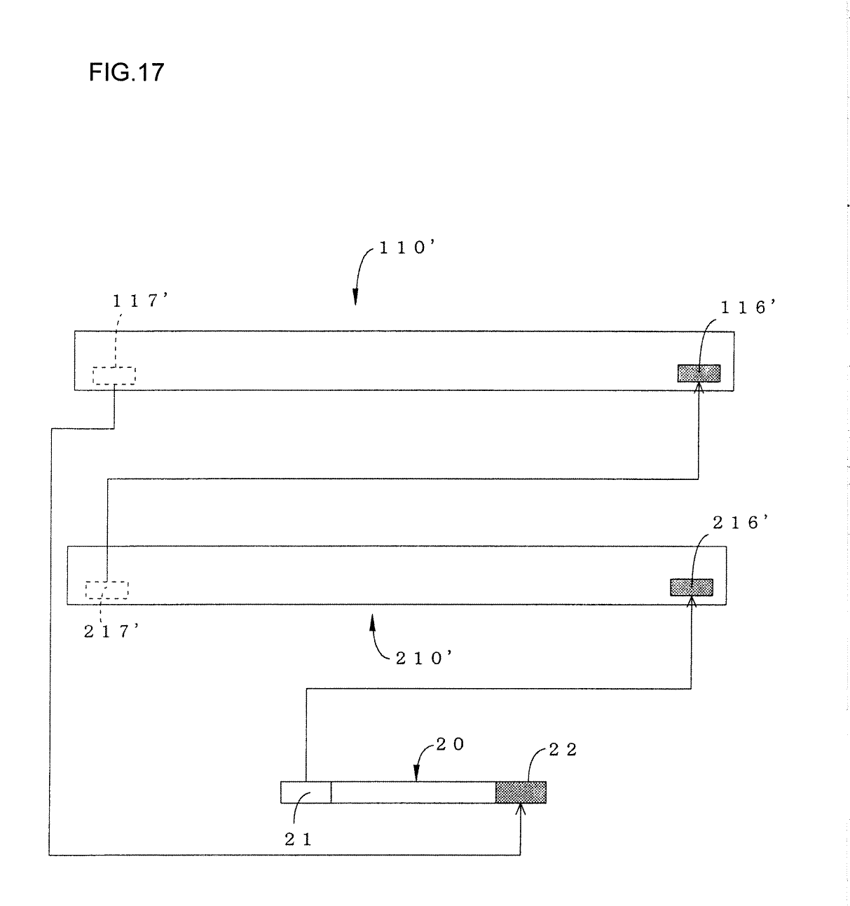

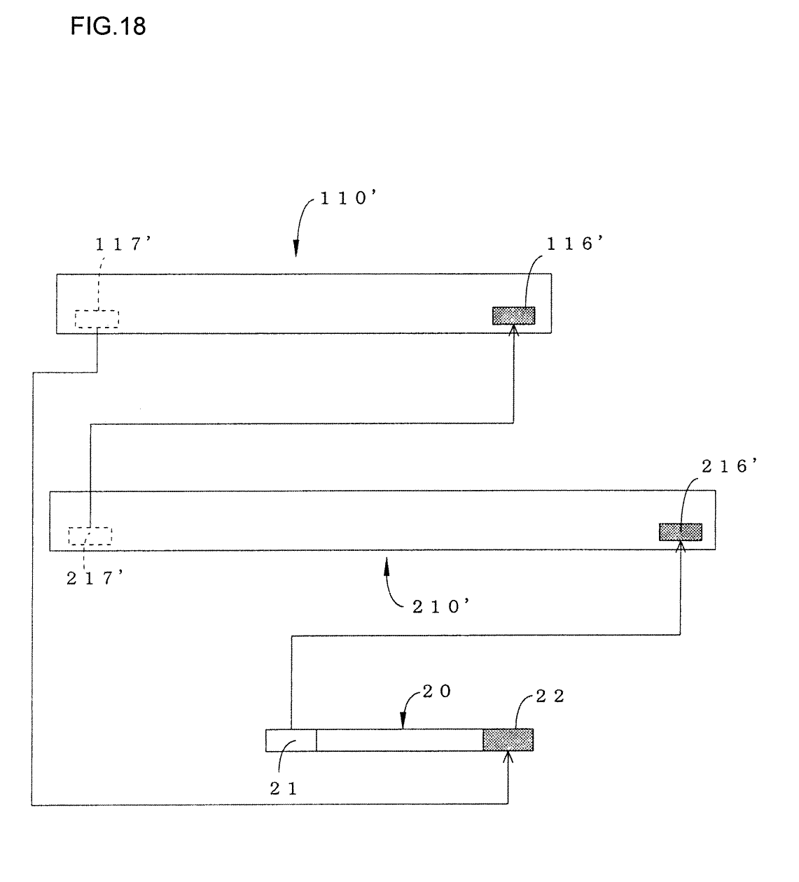

The present embodiment includes, as illustrated in FIGS. 17 and 18, an aspect in which the first water-absorbent cloth 110′, the second water-absorbent cloth 210′, and the elastic member 20 freely attachable/detachable to/from both ends of the first water-absorbent cloth 110′, and freely attachable/detachable to/from both ends of the second water-absorbent cloth 210′ are included, the both ends of the first water-absorbent cloth 110′ are freely attachable/detachable to/from the both ends of the second water-absorbent cloth 210′, and any of usage of attaching the both ends of the elastic member 20 to the both ends of the first water-absorbent cloth 110′, usage of attaching the both ends of the elastic member 20 to the both ends of the second water-absorbent cloth 210′, usage of attaching the both ends of the second water-absorbent cloth 210′ to the both ends of the first water-absorbent cloth 110′, and usage of attaching one end of the first water-absorbent cloth 110′ to the other end of the second water-absorbent cloth 210′, attaching the other end of the first water-absorbent cloth 110′ to the other end of the elastic member 20, and attaching one end of the second water-absorbent cloth 210′ to one end of the elastic member 20 is selected. In the usage using the elastic member 20, of such types of usage, when the body cooling tool is wound around the predetermined portion of the body, the first water-absorbent cloth 110′, the second water-absorbent cloth 210′, or both the first water-absorbent cloth 110′ and the second water-absorbent cloth 210′ are pressed against the predetermined portion of the body by the elastic force of the elastic member 20, whereby the cooling efficiency can be enhanced.

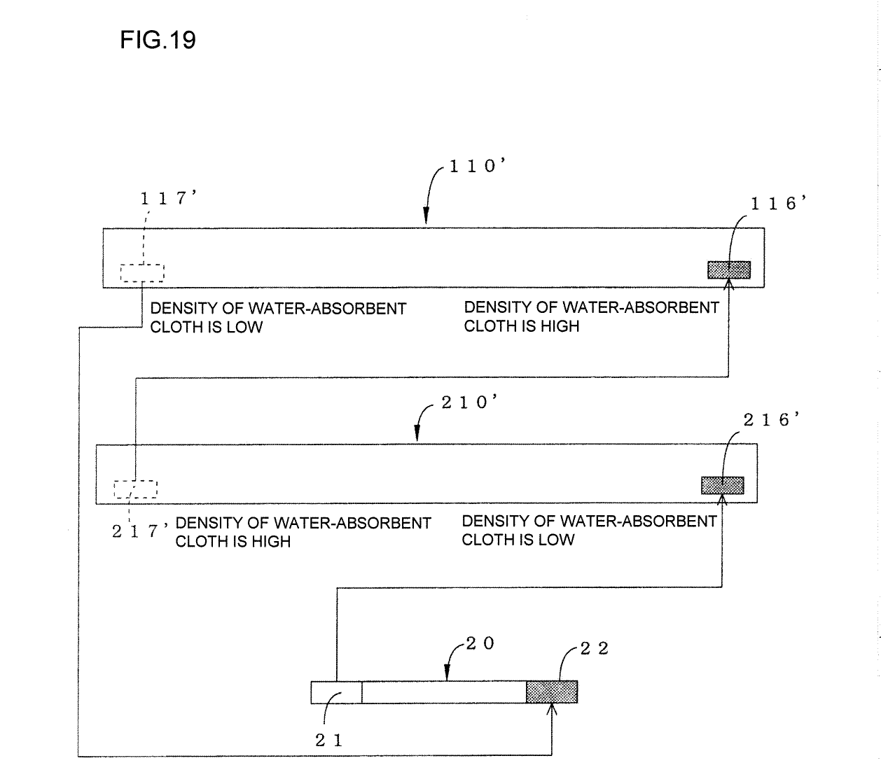

Further, the aspect includes, as illustrated in FIG. 19, an aspect in which the density of the first water-absorbent cloth 110′ is higher in the one end side of the first water-absorbent cloth 110′ than in the other end side, the density of the second water-absorbent cloth 210′ is higher in the other end side of the second water-absorbent cloth 210′ than in the one end side, and in the usage of attaching one end of the first water-absorbent cloth 110′ to the other end of the second water-absorbent cloth 210′, attaching the other end of the first water-absorbent cloth 110′ to the other end of the elastic member 20, and attaching one end of the second water-absorbent cloth 210′ to one end of the elastic member 20, the density of the first water-absorbent cloth 110′ in an end portion at the opposite side to the elastic member 20 is higher than the density of the first water-absorbent cloth 110′ in an end portion at the elastic member 20 side, and the density of the second water-absorbent cloth 210′ in an end portion at an opposite side to the elastic member 20 is higher than the density of the second water-absorbent cloth 210′ in an end portion at the elastic member 20 side.

### Third Embodiment

Next, a third embodiment of the present invention will be described.

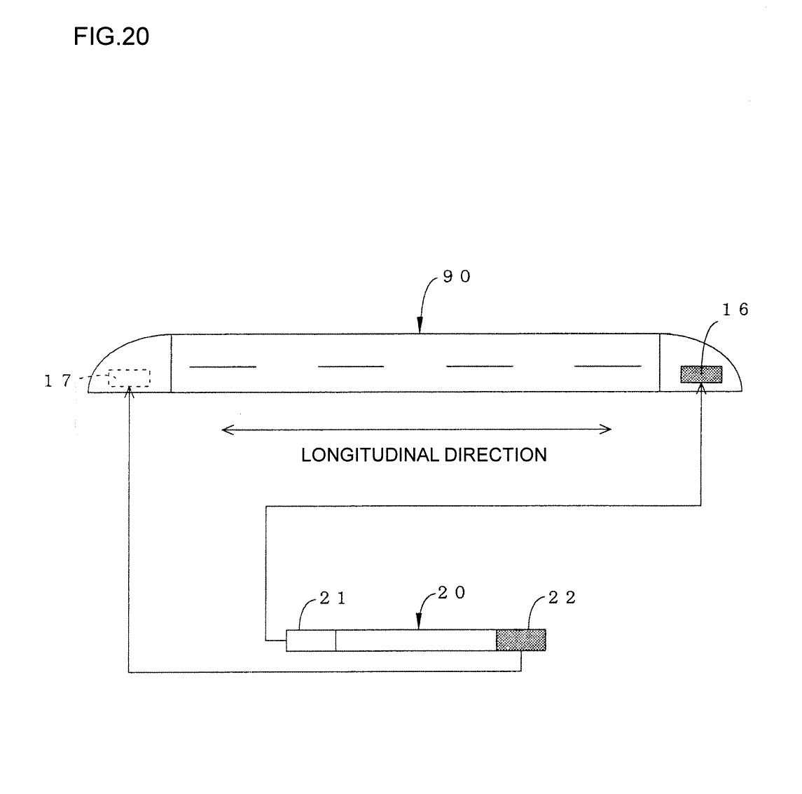

The third embodiment can provide a body warming tool that is wound around a predetermined portion of a body and warms the portion, as illustrated in FIG. 20.

The body warming tool of the present embodiment uses a warming member 90 in place of the above-described cooling member 10, and other configurations are similar to those in the first embodiment. In the third embodiment, the same portion as the first embodiment is denoted with the same reference sign, and detailed description is omitted.

An example of the warming member 90 used in the present embodiment includes one including a warming material and a bag portion 15 that is a narrow long shape and includes the warming material therein. Examples of the warming material include a polymer and a sheet having a heat retaining property. The warming material can have heat by being heated by various means such as a water-bath or a microwave. Further, another example of the warming member 90 used in the present embodiment includes one made of blanket cloth.

According to the present embodiment, when the body warming tool is wound around the predetermined portion of the body, the warming member 90 is pressed against the predetermined portion of the body by elastic force of the elastic member 20, and the density of the warming member 90 is increased, whereby thermal conductivity and heat insulating efficiency can be enhanced.

The present embodiment includes an aspect of employing the “warming member 90” in place of the “cooling member 10” in all the aspects disclosed in the first embodiment. Although detailed description is not given, following aspects are included in the present embodiment.

The present embodiment includes an aspect in which the elastic member 20 is formed in to a band shape with a width of 5 mm or more. The present embodiment includes an aspect in which the elastic member 20 is freely attachable/detachable to/from to the warming member 90. The present embodiment includes an aspect in which an attaching position of the elastic member 20 with respect to the warming member 90 can be changed. The present embodiment includes an aspect in which the length of the elastic member 20 is freely adjustable (see FIG. 4).

The present embodiment includes an aspect in which a first warming member-side attaching portion is provided at one end of an end portion of the warming member 90, a second warming member-side attaching portion is provided at the other end of an end portion of the warming member 90, a first elastic attaching portion 21 is provided at one end of an end portion of the elastic member 20, and a second elastic attaching portion 22 is provided at the other end of an end portion of the elastic member 20, the first warming member-side attaching portion is freely attachable/detachable to/from the first elastic attaching portion 21 and the second warming member-side attaching portion is freely attachable/detachable to/from the second elastic attaching portion 22, and the first warming member-side attaching portion is also freely attachable/detachable to/from the second warming member-side attaching portion.

The present embodiment includes an aspect in which an auxiliary elastic member 40 having a different length from the elastic member 20 is further included (see FIG. 6).

The present embodiment includes an aspect in which notches 18 are provided at predetermined portions of the warming member 90 and the warming member 90 can be bent in a direction perpendicular to a longitudinal direction of the warming member 90 with the notches 18, and the warming member 90 cools at least a back side of a neck of a human body and can pass above ears of the human body.

Further, the present embodiment includes coupling tools 19a′ and 19b′ are provided at predetermined portions of the warming member 90, two places of the warming member 90 are coupled with the coupling tools 19a′ and 19b′ so that the warming member 90 can be bent in the direction perpendicular to the longitudinal direction of the warming member 90, and the warming member 90 cools at least the back side of the neck of the human body and can pass above the ears of the human body.

The present embodiment includes an aspect in which the first warming member, the second warming member, and the elastic member 20 freely attachable/detachable to/from both ends of the first warming member and also freely attachable/detachable to/from both ends of the second warming member are included, the both ends of the first warming member is also freely attachable/detachable to/from the both ends of the second warming member, and any of usage of attaching the both ends of the elastic member 20 to the both ends of the first warming member, usage of attaching the both ends of the elastic member 20 to the both ends of the second warming member, usage of attaching the both ends of the second warming member to the both ends of the first warming member, and usage of attaching one end of the first warming member to the other end of the second warming member, attaching the other end of the first warming member to the other end of the elastic member 20, and attaching one end of the second warming member to one end of the elastic member 20 is selected. In the usage using the elastic member 20, of such types of usage, when the body warming tool is wound around the predetermined portion of the body, the first warming member, the second warming member, or both the first warming member and the second warming member are pressed against the predetermined portion of the body by the elastic force of the elastic member 20, whereby the thermal conductivity and the heat insulating efficiency can be enhanced.

Further, the aspect includes an aspect in which the density of the first warming member is higher in one end side of the first warming member than the other end side, the density of the second warming member is higher in the other end side of the second warming member than one end side, and in the usage of attaching one end of the first warming member to the other end of the second warming member, attaching the other end of the first warming member to the other end of the elastic member 20, and attaching one end of the second warming member to one end of the elastic member 20, the density of the first warming member in an end portion at an opposite side to the elastic member 20 is higher than the density of the first warming member in an end portion at the elastic member 20 side, and the density of the second warming member in an end portion at an opposite side to the elastic member 20 is higher than the density of the second warming member in an end portion at the elastic member 20 side.

Finally, the above-description of the embodiments, the disclosure of the modification and the drawings are mere examples for describing the invention written in claims, and the invention written in claims is not limited by the description of the embodiments and the disclosure of the drawings.

### Reference Signs List

10 Cooling member
11 Water-absorbent material
15 Bag portion
16 First bag attaching portion (first cooling member attaching portion)
17 Second bag attaching portion (second cooling member attaching portion)
18 Notch
19a and 19b Bag coupling tool (coupling tool)
19a′ and 19b′ Coupling tool
21 First elastic attaching portion
22 Second elastic attaching portion
25 Adjustment portion
40 Auxiliary elastic member
41 First auxiliary elastic attaching portion
42 Second auxiliary elastic attaching portion
90 Warming member
110 First cooling member
111 First water-absorbent material
115 First bag portion
116 Attaching portion of one end of first cooling member
117 Attaching portion of the other end of first cooling member
210 Second cooling member
211 Second water-absorbent material
215 Second bag portion
216 Attaching portion of one end of second cooling member
217 Attaching portion of the other end of second cooling member

We claim

1. A body cooling tool which is wound around a predetermined portion of a body and which cools the portion comprising:
a cooling member being a narrow long shape and having a water-absorbent material; and
an elastic member provided at an end portion of the cooling member,
wherein the water-absorbent material is expanded when moisture is contained in the water-absorbent material, and when the moisture contained in the water-absorbent material is vaporized, heat is drawn so that a cooling function is realized, and
wherein when the body cooling tool is wound around the predetermined portion of the body, the cooling member is pressed against the predetermined portion of the body by elastic force of the elastic member, and the density of the water-absorbent material is increased so that cooling efficiency is enhanced.

2. The body cooling tool according to claim 1 further comprises a bag portion including the water-absorbent material therein,
wherein the water-absorbent material is a water-absorbent polymer.

3. The body cooling tool according to claim 1,
wherein the water-absorbent material is water-absorbent cloth.

4. The body cooling tool according to claim 1,
wherein the elastic member is formed into a band shape with a width of 5 mm or more.

5. The body cooling tool according to claim 1,
wherein the elastic member is freely attachable/detachable to/from the cooling member.

6. The body cooling tool according to claim 5,
wherein when predetermined pulling force is added, the elastic member is detached from the cooling member.

7. The body cooling tool according to claim 1,
wherein an attaching position of the elastic member with respect to the cooling member or an attaching position of the cooling member with respect to the elastic member can be changed.

8. The body cooling tool according to claim 1,
wherein the length of the elastic member or the length of the cooling member is freely adjustable.

9. The body cooling tool according to claim 1,
wherein a first cooling member attaching portion is provided at one end of end portions of the cooling member, and a second cooling member attaching portion is provided at the other end of the end portions of the cooling member,
wherein a first elastic attaching portion is provided at one end of end portions of the elastic member, and a second elastic attaching portion is provided at the other end of the end portions of the elastic member,
wherein the first cooling member attaching portion is freely attachable/detachable to/from the first elastic attaching portion, and the second cooling member attaching portion is freely attachable/detachable to/from the second elastic attaching portion, and the first cooling member attaching portion is freely attachable/detachable to/from the second cooling member attaching portion.

10. The body cooling tool according to claim 1 further comprises an auxiliary elastic member having a different length from the elastic member.

11. The body cooling tool according to claim 1,
wherein a notch is provided at a predetermined portion of the cooling member, and the bag portion is able to be bent with the notch in a direction perpendicular to the longitudinal direction of the cooling member, and
wherein the cooling member cools at least the back side of the neck of the human body, and is able to pass above ears of the human body.

12. The body cooling tool according to claim 1,
wherein coupling tools are provided at a predetermined portion of the cooling member, and the two places of the cooling member are coupled with the coupling tools so that the bag portion is able to be bent in the direction perpendicular to the longitudinal direction of the cooling member, and
wherein the cooling member cools at least the back side of the neck of the human body, and is able to pass above ears of the human body.

13. A body cooling tool which is wound around a predetermined portion of a body and which cools the portion comprising:
a first cooling member being a narrow long shape and having a first water-absorbent material; and
a second cooling member being a narrow long shape and having a second water-absorbent material; and
an elastic member being freely attachable/detachable to/from both ends of the first cooling member, and being freely attachable/detachable to/from both ends of the second cooling member;
wherein each the first water-absorbent material and the second water-absorbent material is expanded when moisture is contained in the first water-absorbent material and the second water-absorbent material, and when the moisture contained in the first water-absorbent material and the second water-absorbent material is vaporized, heat is drawn so that a cooling function is realized,
wherein an end of the first cooling member is also freely attachable/detachable to/from both sides of the first cooling member,
wherein any of following types of usage can be selected: usage of attaching both ends of the elastic member to both ends of the first cooling member; usage of the attaching the both ends of the elastic member to both ends of the second cooling member; and usage of attaching the one end of the first cooling member to the other end of the second cooling member, attaching the other end of the first cooling member to the other end of the elastic member, and attaching the one end of the second cooling member to the one end of the elastic member,
wherein when the body cooling tool is wound around the predetermined portion of the body, the first cooling member, the second cooling member or the both of the first cooling member and the second cooling member is pressed against the predetermined portion of the body by elastic force of the elastic member, and the density of the water-absorbent material is increased so that cooling efficiency is enhanced.

14. The body cooling tool according to claim 13,
wherein the density of the first water-absorbent material of the first cooling member is higher in the one end side of the first cooling member than the other end side,
wherein the density of the second water-absorbent material of the second cooling member is higher in the other end side of the second cooling member than the one end side,
wherein in the usage of attaching the one end of the first cooling member to the other end of the second cooling member, attaching the other end of the first cooling member to the other end of the elastic member, and attaching the one end of the second cooling member to the one end of the elastic member, the density of the first water-absorbent material of the first cooling member in the end portion at the opposite side to the elastic member is made higher than the density of the first water-absorbent material of the first cooling member in the end portion at the elastic member side, and the density of the second water-absorbent material of the second cooling member in the end portion at the opposite side to the elastic member is made higher than the density of the second water-absorbent material of the second cooling member in the end portion at the elastic member side.

15. A body warming tool which is wound around a predetermined portion of a body and which warms the portion comprising:
a warming member having a heat retaining property; and
an elastic member provided at an end portion of the warming member,
wherein when body warming tool is wound around the predetermined portion of the body, the warming member is pressed against the predetermined portion of the body by elastic force of the elastic member, and the density of the warming member is increased, and therefore thermal conductivity and heat insulating efficiency is enhanced.
